(19) United States
(12) Patent Application Publication
(10) Pub. No.: US 2025/0213596 A1
(43) Pub. Date: Jul. 3, 2025

(54) CANNABINOID-CONTAINING COMPOSITIONS

(71) Applicant: BUZZELET DEVELOPMENT AND TECHNOLOGIES LTD., Or Akiva (IL)

(72) Inventor: Aharon EYAL, Jerusalem (IL)

(21) Appl. No.: 18/852,519

(22) PCT Filed: Mar. 30, 2023
(86) PCT No.: PCT/IB2023/053185

§ 371 Date: Sep. 30, 2024

Related U.S. Application Data
(60) Provisional application No. 63/326,288, filed on Apr. 1, 2022.
(60) Provisional application No. 63/341,228, filed on May 12, 2022.

(51) Int. Cl. A61K 31/00; A61K 9/14; A61K 9/48; A61K 31/01

(52) U.S. Cl. CPC A61K 31/658; A61K 9/143; A61K 9/485; A61K 31/01

(57) ABSTRACT

Provided is a composition comprising at least one carrier comprising a nanoporous material; and at least one cannabinoid and at least one non-cannabinoid, non-terpene impurity, wherein a concentration of carrier is between about 30 wt % and about 90 wt % of the total composition; a concentration of the cannabinoid is between about 10 wt % and about 70 wt % of the total composition; a combined concentration of the cannabinoid and the carrier is at least about 80 wt % of the total composition; at least about 90 wt % of the cannabinoid is adsorbed on the carrier; a ratio between a total weight of the cannabinoid and a total weight of the at least one impurity is at least about 1.5:1; and the carrier comprises discrete particles. Further provided are methods of preparation and methods of use of the composition.

### FIELD OF THE INVENTION

The present invention relates to the field of cannabinoid-containing compositions, and more specifically to such compositions comprising at least one cannabinoid and a nanoporous carrier.

### BACKGROUND

At about ambient temperatures, i.e. temperatures of less than 50° C., concentrated cannabinoids form highly viscous materials with a consistency such as that of a resin or wax. This viscosity increases with increased purity. As such, pouring concentrated cannabinoids at about ambient temperatures is impractical and any transfer from one vessel to another requires heating, which may affect the chemical stability of the product. Complete transfer is impractical, such that losses of expensive concentrated cannabinoids are unavoidable.

Furthermore, heating leads to decarboxylation of acid forms of cannabinoids, such that the presence of the acids forms is severely reduced, even though these may be the preferred form of the cannabinoids.

One solution known in the art is crystallization of cannabinoids. However, most cannabinoids do not form real crystals. Another known solution involves diluting the cannabinoid in a solvent. However, shipping of solvents is problematic, particularly by air transport. Additionally, suitable solvents, such as vegetable oils are problematic in certain uses, such as vaping. Such oils also interfere with the formation of certain formulations, such as tablets.

There is thus an unmet need for a flowable form of cannabinoids having high levels of purity, which is easy to transfer from one vessel to another, safe to transport, stable and suitable for use by a consumer.

### SUMMARY OF THE INVENTION

According to an aspect of some embodiments of the present invention, there is provided a composition comprising:
(a) at least one carrier, comprising a nanoporous material; and
(b) at least one cannabinoid and at least one non-cannabinoid, non-terpene impurity, wherein
(i) a concentration of the at least one carrier is between 30 wt % and 90 wt % of the total composition;
(ii) a concentration of the at least one cannabinoid is between 10 wt % and 70 wt % of the total composition;
(iii) a combined concentration of the at least one cannabinoid and the at least one carrier is at least 80 wt % of the total composition;
(iv) at least 90 wt % of the at least one cannabinoid is adsorbed on the carrier;
(v) a ratio between a total weight of the cannabinoids and a total weight of the at least one impurity is at least 1.5:1; and
(vi) the carrier comprises discrete particles.

According to a further aspect of some embodiments of the present invention, there is provided a method for the production of the composition as disclosed herein, the method comprising
(i) providing a carrier comprising a nanoporous material;
(ii) providing a source comprising at least one cannabinoid, which source is selected from the group consisting of cannabis plant materials, fermentation products and bio-reactor products;
(iii) extracting the source with a solvent to form an extract comprising the at least one cannabinoid and at least one non-cannabinoid, non-terpene impurity;
(iv) removing the impurity from the extract to form a purified extract; and
(v) adsorbing the purified extract on the carrier, whereby the composition is formed.

According to a further aspect of some embodiments of the present invention, there is provided a method for transporting a purified cannabinoid, the method comprising providing the composition as disclosed herein, wherein the at least one cannabinoid is the purified cannabinoid having a purity greater than 90%; and transporting the composition.

According to a further aspect of some embodiments of the present invention, there is provided a method for transporting a purified cannabinoid, the method comprising
(i) providing at least one carrier, comprising a nanoporous material;
(ii) providing at least one purified cannabinoid to be transported, the purified cannabinoid having a purity greater than 90%,
(iii) adsorbing the purified cannabinoid on the cannabinoid, wherein a carrier-cannabinoid composition is formed; and
(iv) transporting the carrier-cannabinoid composition.

### DETAILED DESCRIPTION OF THE INVENTION

The present invention relates to compositions comprising at least one cannabinoid and a nanoporous carrier.

The invention provides a flowable form of cannabinoids having high purity (such as at least 90% or at least 95%), which is easily transferable from one vessel to another, easily transportable, stable, and suitable for direct use by a consumer or for use in the preparation of other formulations, such as specific dosage forms.

As used herein, the term “flowable” with regard to a composition is intended to mean that a majority of particles of the composition have the ability to move freely relative to an inner surface of a vessel in which they are contained.

Unless otherwise defined, all technical and scientific terms used herein have the same meaning as commonly understood by one of ordinary skill in the art to which this invention belongs.

The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a composition comprising:
(a) at least one carrier, comprising a nanoporous material; and
(b) at least one cannabinoid and at least one non-cannabinoid, non-terpene impurity, wherein
   (i) a concentration of the at least one carrier is between about 30 wt % and about 90 wt % of the total composition;
   (ii) a concentration of the at least one cannabinoid is between about 10 wt % and about 70 wt % of the total composition;
   (iii) a combined concentration of the at least one cannabinoid and the at least one carrier is at least about 80 wt % of the total composition;
   (iv) at least about 90 wt % of the at least one cannabinoid is adsorbed on the carrier;
   (v) a ratio between a total weight of the cannabinoids and a total weight of the at least one impurity is at least about 1.5:1; and
   (vi) the carrier comprises discrete particles.

As used herein, the term “nanoporous” refers to a material comprising pores having a diameter in the nanometer range i.e. in the range of from about 1 nm to about 999 nm, such as about 1 nm, about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm or up to about 999 nm. According to some such embodiments, the pores have a diameter in the range of from about 0.5 to about 200 nm. According to some embodiments, the material is mesoporous, i.e. having pores of a diameter in the range of from about 2 to about 50 nm.

According to some embodiments, the carrier is selected from the group consisting of silica, zeolite, granular active carbon and combinations thereof. According to some such embodiments, the material is mesoporous silica.

According to some embodiments, an internal surface of the at least one carrier is lipophilic.

According to some embodiments, an internal surface of the at least one carrier is hydrophilic.

According to some embodiments, a concentration of the at least one carrier is about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, or about 90 wt % of the total composition.

According to some embodiments, the at least one carrier has a surface area between about 50 m²/gr and about 1000 m²/gr, such as about 50 m m²/gr, about 60 m²/gr, about 70 m m²/gr, about 80 m²/gr, about 90 m²/gr, about 100 m²/gr, about 200 m²/gr, about 300 m²/gr, about 400 m²/gr, about 500 m²/gr, about 600 m²/gr, about 700 m²/gr, about 800 m²/gr, about 900 m²/gr, or about 1000 m²/gr.

According to some embodiments, the composition comprises one, two, three, four or more than four cannabinoids.

According to some embodiments, the at least one cannabinoid is selected from the group consisting of tetrahydrocannabinolic acid (THCA), tetrahydrocannabinol (THC), cannabidiolic acid (CBDA), cannabidiol (CBD), cannabigerolic acid (CBGA), cannabigerol (CBG), cannabichromenic acid (CBCA), cannabichromene (CBC), tetrahydrocannabivarinic acid (THCVA), tetrahydrocannabivarin (THCV), cannabidivarinic acid (CBDVA), cannabidivarin (CBDV) and combinations thereof and combinations thereof.

According to some embodiments, a total concentration of the cannabinoids is about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, or about 70 wt % of the total composition.

According to some embodiments, the at least one cannabinoid comprises at least two cannabinoids, wherein a first of the at least two cannabinoids is present in acid form and a second of the at least two cannabinoids is present in decarboxylated form and wherein the weight/weight ratio between the total amount of cannabinoids in acid form and the total amount of cannabinoids in decarboxylated form is greater than about 2:1. According to some embodiments, the weight/weight ratio between the total amount of cannabinoids in acid form and the total amount of cannabinoids in decarboxylated form is up to about 1000:1. According to some embodiments, the weight/weight ratio between the total amount of cannabinoids in acid form and the total amount of cannabinoids in decarboxylated form is about 3:1, about 5:1, about 10:1, about 50:1, about 100:1, about 200:1, about 300:1, about 400:1, about 500:1, about 600:1, about 700:1, about 800:1, about 900:1 or about 1000:1.

According to some embodiments, a combined concentration of the at least one cannabinoid and the at least one carrier is about 80 wt %, about 85 wt %, about 86 wt %, about 87 wt %, about 88 wt %, about 89 wt %, 90 wt %, about 91 wt %, about 92 wt %, about 93 wt %, about 94 wt %, about 95 wt %, about 96 wt %, about 97 wt %, about 98 wt %, about 99 wt %, or about 100 wt % of the total composition.

According to some embodiments, the non-terpene impurity is an impurity such as waxes, chlorophyl, chlorophyl degradation products and suspended sub-micron particles, each of which may be independently present in an amount of up to about 2% wt of the total composition.

According to some embodiments, about 90 wt %, about 91 wt %, about 92 wt %, about 93 wt %, about 94 wt %, about 95 wt %, about 96 wt %, about 97 wt %, about 98 wt %, about 99 wt %, or about 100 wt % of the at least one cannabinoid is adsorbed on the carrier.

According to some embodiments, a ratio between a total weight of the cannabinoids and a total weight of the at least one impurity is about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 10.5:1, about 11:1, about 11.5:1, about 12:1, about 12.5:1, about 13:1, about 13.5:1, about 14:1, about 14.5:1, about 15:1, about 20:1, about 50:1, about 100:1, about 200:1, about 500:1, about 1000:1, about 2000:1, about 3000:1, about 4000:1, about 5000:1, about 600:1, about 7000:1, about 8000:1, about 9000:1, or about 1000:1.

As used herein, the term “distinct particles” refers to individual particles, each having a distinct shape.

According to some embodiments, the composition further comprises at least one terpene, such as one, two, three, four, five, six, seven or eight terpenes. According to some such embodiments, the at least one terpene is selected from the group consisting of pinene, limonene, linalool, myrcene, borneol, terpineol, geraniol, eucalyptol, terpinolene, sabinene,, ocimene, terpinene, caryophyllene, humulene, nerolidol, guaiol, bisabolol and combinations thereof.

According to a further aspect of some embodiments of the present invention, there is provided a product comprising at least a first composition as disclosed herein, wherein the product is selected from the group consisting of tablets, gel capsules, vaping materials, smokable materials, cigarettes, suppositories, foods and beverages.

According to some embodiments, the product comprises at least two compositions as disclosed herein, wherein a first of the at least two compositions is different from a second of the at least two compositions. According to some such embodiments, the first and the second of the at least two compositions differ in at least one selected from the group consisting of the specific cannabinoids present, the concentrations of the cannabinoids present, the cannabinoid: carrier ratios of the cannabinoids present, and combinations thereof.

According to some embodiments, the product further comprises a second composition comprising a second carrier and at least one second cannabinoid. According to some such embodiments, the second carrier comprises a nanoporous material. According to some embodiments, the at least one second cannabinoid is selected from the group consisting of tetrahydrocannabinolic acid (THCA), tetrahydrocannabinol (THC), cannabidiolic acid (CBDA), cannabidiol (CBD), cannabigerolic acid (CBGA), cannabigerol (CBG), cannabichromenic acid (CBCA), cannabichromene (CBC), tetrahydrocannabivarinic acid (THCVA), tetrahydrocannabivarin (THCV), cannabidivarinic acid (CBDVA), cannabidivarin (CBDV) and combinations thereof

According to some embodiments, the product further comprises a third composition comprising a third carrier and at least one second terpene. According to some such embodiments, the third carrier comprises a nanoporous material. According to some embodiments, the at least one second terpene is selected from the group consisting of pinene, limonene, linalool, myrcene, borneol terpineol, geraniol, eucalyptol, terpinolene, sabinene, ocimene, terpinene, caryophyllene, humulene, nerolidol, guaiol, bisabolol and combinations thereof.

According to some embodiments, the product further comprises a second carrier and at least one second cannabinoid; and a third carrier and at least one second terpene.

According to a further aspect of some embodiments of the present invention, there is provided a method for the production of the composition as disclosed herein, the method comprising:
(i) providing a carrier comprising a nanoporous material;
(ii) providing a source comprising at least one cannabinoid, which source is selected from the group consisting of cannabis plant materials, fermentation products and bio-reactor products;
(iii) extracting the source with a solvent to form an extract comprising the at least one cannabinoid and at least one non-cannabinoid, non-terpene impurity;
(iv) removing the impurity from the extract to form a purified extract; and
(v) adsorbing the purified extract on the carrier, whereby the composition is formed.

According to some embodiments, removing the impurity is performed by a method selected from the group consisting of chromatographic separation, distillation, heating, adsorption, crystallization and combinations thereof.

According to some embodiments, the method further comprises adsorbing at least one additional cannabinoid and/or at least one terpene on the carrier.

According to a further aspect of some embodiments of the present invention, there is provided a method for transporting a purified cannabinoid, the method comprising
(i) providing the composition as disclosed herein, wherein the at least one cannabinoid is the purified cannabinoid having a purity greater than 90%; and
(ii) transporting the composition.

According to some embodiments, the method further comprises recovering at least a fraction of the at least one cannabinoid from the composition. According to some such embodiments, recovering comprises at least one selected from the group consisting of distilling at a pressure of less than about 10 millibar (such as less than about 10 millibar, about 9 millibar, about 8 millibar, about 7 millibar, about 6 millibar, about 5 millibar, about 4 millibar, about 3 millibar, about 2 millibar, about 1 millibar, about 0.5 millibar, about 0.1 millibar, about 0.05 millibar, about 0.01 millibar, about 0.005 millibar, or even less than about 0.001 millibar); contacting with an organic solvent; contacting with water or with an aqueous solution; and desorption.

According to an aspect of some embodiments of the present invention, there is provided a method for transporting a purified cannabinoid, the method comprising
(i) providing at least one carrier, comprising a nanoporous material;
(ii) providing at least one purified cannabinoid to be transported, the purified cannabinoid having a purity greater than 90%,
(iii) adsorbing the purified cannabinoid on the cannabinoid, wherein a carrier-cannabinoid composition is formed; and
(iv) transporting the carrier-cannabinoid composition.

According to some embodiments, the method further comprises recovering at least a fraction of the at least one cannabinoid from the composition. According to some such embodiments, recovering comprises at least one selected from the group consisting of distilling at a pressure of less than 10 millibar (such as less than about 10 millibar, about 9 millibar, about 8 millibar, about 7 millibar, about 6 millibar, about 5 millibar, about 4 millibar, about 3 millibar, about 2 millibar, about 1 millibar, about 0.5 millibar, about 0.1 millibar, about 0.05 millibar, about 0.01 millibar, about 0.005 millibar, or even less than about 0.001 millibar); contacting with an organic solvent; contacting with water or with an aqueous solution; and desporption.

According to a further aspect of some embodiments of the present invention, there is provided a method for the production of a product comprising at least two compositions as disclosed herein, comprising blending the at least two compositions.

According to some embodiments, there is provided a method for the production of a product comprising at least a first composition as disclosed herein and a second composition comprising a second carrier and at least one second cannabinoid, the method comprising blending the first composition with the second composition.

According to some embodiments, there is provided a method for the production of a product comprising at least a first composition as disclosed herein, and a third composition comprising a third carrier and at least one second terpene, the method comprising blending the first composition with the third composition.

According to some embodiments, there is provided a method for the production of a product comprising the first composition as disclosed herein; a second composition comprising a second carrier and at least one second cannabinoid; and a third composition comprising a third carrier and at least one second terpene, the method comprising blending the first composition with the second composition and with the third composition.

According to some embodiments of the present invention the product is in a smokable form. According to such some embodiments the product comprises a smokable material. According to such some embodiments the smokable material is selected from the group consisting of cannabis, pre-extracted cannabis, tobacco and combinations thereof. According to some embodiments the product is provided in a form selected from the group consisting of joints, cigarettes, blunts, bongs, bubbkers, hookah and nargilla. According to some such embodiments, the product is a cigarette.

According to a further aspect of some embodiments of the present invention there is provided a method for producing a cannabinoid-enriched smokable material, the method comprising blending a smokable material with at least one composition as disclosed herein.

According to a further aspect of some embodiments of the present invention there is provided a cigarette comprising the cannabinoid-enriched smokable material as disclosed herein.

According to a further aspect of some embodiments of the present invention, there is provided a method of providing a cannabinoid to a subject comprising administering to said subject a composition as disclosed herein.

According to a further aspect of some embodiments of the present invention, there is provided a method of providing a cannabinoid to a subject comprising administering to said subject a product as disclosed herein.

### EXAMPLES

### Example 1

Purified THC at 99% purity (dronabinol) was used. It was highly viscous and could not be poured out of its container.

5 gr of the dronabinol were dissolved in 5 gr of 95% ethanol. Full dissolution was observed and a suspension-free ethanol solution was formed.

10 gr of the ethanol solution were mixed with 10 gr of Grace® Syloid® XDP3050 silica particles. The solution was completely adsorbed on the silica particles.

The solution-carrying particles were kept at ambient temperature overnight for ethanol evaporation.

A product comprising 66% silica and 33% THC was formed. It felt dry and was free flowing. It transferred fully from one vessel to another.

### Example 2

The procedure of Example 1 was repeated, except that 10 gr of the ethanol solution were mixed with 20 gr of the silica particles. A free-flowing product was formed comprising about 80% silica and about 20% THC.

### Examples 3 and 4

The procedures of Examples 1 and 2 were repeated, this time using crystalline CBD isolate instead of dronabinol. CBD concentration in the isolate was about 98%. Free-flowing products were formed comprising 66% silica and 33% CBD (Example 3) and comprising about 80% % silica and about 20% CBD (Example 4).

### Example 5

10 gr of ground cannabis inflorescence were mixed with 100 gr of 95% ethanol for 30 minutes at ambient temperature. The residual plant material was then removed by filtration and the remaining ethanol solution was treated in a rotovapor for complete removal of the ethanol. An ethanol-free extract was formed, containing about 45% THCA and about 28% CBDA.

10 gr of the extract were mixed with 10 gr of the silica particles. The extract was fully adsorbed and a free-flowing product was formed, comprising 60% silica, 18% THCA and 11% CBDA.

### Example 6

6 gr of dronabinol were mixed with 2 gr medium-chain triglyceride (MCT) and the mixture was heated to 80° C. to form a THC-MCT liquid.

The formed liquid was mixed with 10 gr of the silica particles. The liquid was fully adsorbed and a free-flowing product was formed, comprising 55% silica and 33% THCA.

### Example 7

Gel capsules are filled with 100 mg particles formed according to Example 2. A gel capsule containing 20 mg THC is formed.

### Example 8

Gel capsules are filled with 50 mg particles formed according to Example 3. A gel capsule containing 16 mg CBD is formed.

### Example 9

Gel capsules are filled with 30 mg particles formed according to Example 1 and 40 mg particles formed according to Example 3. A gel capsule containing 10 mg THC and 8 mg CBD is formed.

### Example 10

2 gr of particles produced according to Example 1, were suspended in 20 gr ethanol of 95%. The suspension was kept at 40 C° for 20 min in a sonication bath. The solid particles were then removed by filtration. Analysis of the ethanol solution showed that more than 90% of the adsorbed THC was extracted into the ethanol solution.

### Example 11

1 gr of particles produced according to Example 1, were suspended in 25 gr of 2% SLS aqueous solution. The suspension was stirred for 2 hours at 37° C. The solid particles were then removed by filtration. Analysis of the SLS solution showed that more than 80% of the adsorbed THC was liberated.

1. A composition comprising:
(a) at least one carrier, comprising a nanoporous material; and
(b) at least one cannabinoid and at least one non-cannabinoid, non-terpene impurity, wherein
(i) a concentration of said at least one carrier is between 30 wt % and 90 wt % of the total composition;
(ii) a concentration of said at least one cannabinoid is between 10 wt % and 70 wt % of the total composition;
(iii) a combined concentration of said at least one cannabinoid and said at least one carrier is at least 80 wt % of the total composition;
(iv) at least 90 wt % of said at least one cannabinoid is adsorbed on said carrier;
(v) a ratio between a total weight of said cannabinoids and a total weight of said at least one impurity is at least 1.5:1; and
(vi) said carrier comprises discrete particles.

2-3. (canceled)

4. The composition of claim 1, wherein said at least one cannabinoid comprises at least two cannabinoids, wherein a first of said at least two cannabinoids is present in acid form and a second of said at least two cannabinoids is present in decarboxylated form.

5-6. (canceled)

7. The composition of claim 1, wherein said at least one carrier comprises at least one selected from the group consisting of silica, zeolite, granular active carbon and combinations thereof.

8. (canceled)

9. The composition of claim 1, wherein an internal surface of said at least one carrier is hydrophilic.

10. A product comprising at least a first composition according to claim 1, wherein the product is selected from the group consisting of tablets, gel capsules, vaping materials, smokable materials, cigarettes, suppositories, foods and beverages.

11. A product comprising at least two compositions according to claim 1, wherein the product is selected from the group consisting of tablets, gel capsules, vaping materials, smokable materials, cigarettes, suppositories, foods and beverages, and wherein a first of said at least two compositions is different from a second of said at least two compositions.

12. The product of claim 10, further comprising a second composition comprising a second carrier and at least one second cannabinoid.

13-14. (canceled)

15. The product of claim 12, further comprising a third composition comprising a third carrier and at least one second terpene.

16-17. (canceled)

18. The product according to claim 10, further comprising a second carrier and at least one second cannabinoid; and a third carrier and at least one second terpene.

19. A method for the production of the composition of claim 1 comprising
(i) providing a carrier comprising a nanoporous material;
(ii) providing a source comprising at least one cannabinoid, which source is selected from the group consisting of cannabis plant materials, fermentation products, synthetic products, and bio-reactor products;
(iii) extracting said source with a solvent to form an extract comprising said at least one cannabinoid and at least one non-cannabinoid, non-terpene impurity;
(iv) removing said impurity from said extract to form a purified extract; and
(v) adsorbing said purified extract on said carrier, whereby said composition is formed.

20. The method of claim 19, wherein removing said impurity is performed by a method selected from the group consisting of chromatographic separation, distillation, heating, adsorption, crystallization and combination thereof.

21. (canceled)

22. A method for transporting a purified cannabinoid, the method comprising
(i) providing the composition of claim 1, wherein said at least one cannabinoid is the purified cannabinoid having a purity greater than 90%; and
(ii) transporting said composition.

23. The method of claim 22, further comprising recovering at least a fraction of said at least one cannabinoid from said composition.

24. The method of claim 23, wherein said recovering comprises at least one selected from the group consisting of distilling at a pressure of less than 10 millibar, contacting with an organic solvent, contacting with water or with an aqueous solution and desorption.

25-31. (canceled)

32. The product of claim 10, comprising a smokable material.

33. The product of claim 32, wherein said smokable material is selected from the group consisting of cannabis, pre-extracted cannabis, tobacco and combinations thereof.

34-37. (canceled)

38. A method of providing a cannabinoid to a subject comprising administering to said subject a composition according to claim 1.

39. A method of providing a cannabinoid to a subject comprising administering to said subject a product according to claim 10.
